(19) United States
(12) Patent Application Publication
(10) Pub. No.: US 2014/0271766 A1
(43) Pub. Date: Sep. 18, 2014

(54) METHODS AND DEVICE FOR TREATING OPIOID ADDICTION

(71) Applicant: Titan Pharmaceuticals, Inc., South San Francisco, CA (US)

(72) Inventor: Rajesh Patel, Redwood City, CA (US)

(73) Assignee: Titan Pharmaceuticals, Inc., South San Francisco, CA (US)

(21) Appl. No.: 14/211,584

(22) Filed: Mar. 14, 2014

Related U.S. Application Data
(60) Provisional application No. 61/799,224, filed on Mar. 15, 2013.

(51) Int. Cl. A61K 9/51; A61K 31/485

(52) U.S. Cl. CPC A61K 9/5138; A61K 31/485
USPC 424/422; 514/279

(57) ABSTRACT

The invention described herein solves the challenges encountered in providing a safe, efficacious, and satisfactory option for the treatment of opioid addiction. Methods and devices of the invention allow a subject to receive an implantable formulation comprising an opioid receptor ligand, buprenorphine, or a metabolite thereof as a treatment for opioid addiction. The invention preempts several difficulties encountered with conventional methods for the treatment of opioid addiction, and by doing so the invention improves treatment adherence, compliance, patient satisfaction, and overall success rate.

### CROSS REFERENCE

This application claims priority to U.S. Provisional Application No. 61/799,224, filed on Mar. 15, 2013, the contents of which is incorporated by reference in its entirety.

### BACKGROUND

Dependence on opioids, in the form of heroin or prescription pain medications, is a significant health concern. Methadone maintenance treatment for opioid dependence reduces morbidity, mortality, and the spread of infectious diseases but is restricted to licensed specialty clinics in the United States, requires frequent clinic visits, and has a high risk of overdose. These issues have led to increased use of buprenorphine as a treatment for opioid addiction, and numerous studies support the efficacy of sublingually administered buprenorphine. In the United States, buprenorphine can be prescribed in office based physician practice. However, there are several concerns about diversion and nonmedical use of sublingual buprenorphine. Poor treatment adherence, resulting in craving and withdrawal symptoms that increase the likelihood of relapse, is also a concern with sublingual buprenorphine.

### SUMMARY OF THE INVENTION

In some embodiments, the invention provides a method of treating hydrocodone addiction in a subject in need of relief thereof, the method comprising implanting into a subdermal tissue of the subject a device comprising at least one opioid receptor ligand and a polymer matrix, and subsequently sublingually administering the opioid receptor ligand to the subject, wherein the device releases a therapeutically-effective amount of the opioid receptor ligand.

### BRIEF DESCRIPTION OF THE FIGURES

FIG. 1 Illustrates the plasma pharmacokinetics of the mean buprenorphine concentration after device insertion for subjects with 4 or 5 implants in two distinct studies (triangled and circled line markers).

FIG. 2 Illustrates the plasma pharmacokinetics of the mean buprenorphine concentration after device insertion for subjects with 4 or 5 implants in an open-label study (triangled line marker), and in a double blind study (diamond line marker).

FIG. 3 Is a schematic of the treatment protocol used in two open label treatment studies.

FIG. 4 Illustrates a representative site of implantation of a device of the invention in a human.

FIG. 5 Illustrates implantation of a device in the subdermal tissue of a subject.

FIG. 6 Illustrates the specifications of a representative applicator used to insert a device of the invention into a subject.

FIG. 7 Illustrates the mean and individual plasma buprenorphine concentrations versus time in all subjects receiving four Probuphine implants without receiving supplemental (rescue) sublingual buprenorphine (semi-logarithmic scale).

FIG. 8 Illustrates the mean and individual plasma norbuprenorphine concentrations versus time in all subjects receiving four Probuphine implants without receiving supplemental (rescue) sublingual buprenorphine (semi-logarithmic scale).

### DETAILED DESCRIPTION

Heroin, morphine, and some prescription painkillers, for example, OxyContin, Vicodin, and Fentanyl, belong to a class of drugs known as opiates. They act on specific opiate receptors in the brain, which also interact with naturally produced substances known as endorphins or enkephalins, which are important in regulating pain and emotion. While prescription painkillers are highly beneficial medications when used as prescribed, opiates as a general class of drugs have noteworthy abuse liability. The treatment of opioid addiction represents a significant clinical and societal challenge, and some of the problematic consequences of opioid addiction are characterized by biological, psychological, and social difficulties.

Several attempts have been made to provide efficacious and safe treatments for opioid addiction, many of which are widely described in the literature. Existing treatments include the prescription of methadone, buprenorphine, naltrexone, diamorphine, and levacetylmethanol. However, strict adherence to pharmacological dosage regiments is a pre-requisite to the success of most treatments, and an inherent challenge exists when one prescribes drugs to an individual seeking treatment for substance abuse. Not surprisingly, many existing treatments have only achieved limited success. Compliance is low due to the need for frequent dosing and variable blood levels of the drugs used in the treatment cause withdrawal and cravings, which can lead to a potential relapse.

In an attempt to address some of the existing limitations of pharmacological treatments for opioid dependence, principally medication diversion, implantable devices have been proposed in the art that could alleviate such problems. Nevertheless, existing devices lack features that can provide therapeutically-effective plasma concentrations of the active drug while providing a satisfactory delivery mechanism for long-term usage.

Furthermore, to prevent abuse from implantable devices one must limit the dosages of the active compounds contained within the device, and one must craft a device with an increased breaking strength (resistance to crushing and tearing). A useful implantable device for the treatment of opioid addiction comprises particulates of a compound that is therapeutically-effective and innocuous. The size of the particulates and their plasma rate-of-release are useful properties in crafting a device with an ideal drug dissolution profile and minimal potential for misuse.

The device and methods of the invention provide a treatment regimen of opioid dependence that is a significant departure from existing treatments. The invention achieves statistically significant improvement on adherence to prescribed treatment, non-diversion, and nonmedical uses over the existing treatments designed to target opioid addiction (Example 3). A major advantage of the implantable formulation of the invention is limiting the possibility that very same devices intended for treatment, can be diverted to recreational uses.

In some embodiments, the method provided by the invention comprises a treatment regimen for treating opioid addiction in a subject in need or want of relief thereof, the method consisting of implanting a device comprising a particulate of at least one opioid receptor ligand, and a polymer matrix, to the subject, wherein the device is selected based on the therapeutic effects of the opioid receptor ligand, and wherein the device releases a therapeutically-effective amount of the opioid receptor ligand.

The present invention allows for the selection of the most effective opioid receptor ligand in a specific clinical case. No longer do clinicians and patients need to be limited by existing treatments, and no longer do clinicians have to remove patients from a prescribed opioid addiction treatment if the patient has an adverse affect to one particular drug. The invention has been devised to incorporate dosage forms of opioid receptor ligands in a manner that provide a therapeutically effective dosage of treatment. In some embodiments, the opioid receptor ligand is buprenorphine. In some embodiments, the opioid receptor ligand is norbuprenorphine.

Furthermore, the invention has been devised to provide a therapeutically-effective plasma concentration of buprenorphine, norbuprenorphine, an opioid receptor ligand or a pharmaceutically-acceptable salt of such compounds. Examples of therapeutically-effective plasma concentrations of a device of the invention are illustrated in Examples 2, 3, and 4.

### Methods of the Invention

The methods and device of the invention provide effective, safe, sustainable, and reliable methods for the treatment of opioid addiction. In some embodiments, an implantable device of the invention comprises: buprenorphine or a pharmaceutically-acceptable salt thereof and a polymer matrix, wherein the implantable device has a tensile strength in a range of about 10,000 g/cm² to about 110,000 g/cm², wherein upon implantation in a human the implant releases a therapeutically-effective amount of buprenorphine or the pharmaceutically-acceptable salt thereof to the human.

The present device provides a reduced need for daily supervision and clinical visits, minimizes fluctuations in drug plasma concentrations, improves treatment compliance, and reduces the likelihood of diversion.

Devices of the invention can be packaged as a kit. In some embodiments, a kit includes written instructions on the use of the device. The written material can be, for example, a label. The written material can suggest conditions methods of administration. The instructions provide the subject and the supervising physician with the best guidance for achieving the optimal clinical outcome from the administration of the therapy.

In some embodiments, the particulate is in a solid state. In some embodiments the particulate is a non-crystalline solid that lacks the long-range order characteristic of a crystal, and therefore is present in an amorphous state. Amorphous forms of the particulate include, for example, gels, thin films, and nanostructured materials.

A method of treating opioid addiction in a subject in need or want of relief thereof can comprise implanting a device comprising buprenorphine or a metabolite thereof, and a polymer matrix, to the subject, wherein the device provides a plasma concentration of buprenorphine or the metabolite thereof of about 50 pg/ml to about 4,500 pg/ml over a period of at least about 24 weeks (Example 2). Randomized, double-blind, placebo-controlled multi-center studies of the system and device of the invention in patients with opioid dependence indicate that at least one embodiment of the present invention is statistically superior to the current standard-of-care treatments in helping individuals overcome their dependence on opioids (Example 3).

### Opioids, Metabolites, and Drug Metabolism.

The methods and device described herein are used to treat different types of opioid addiction. In some embodiments, an opioid receptor ligand such as an opiate, a synthetic opioid, a semi-synthetic opioid, a partial opioid agonist, buprenorphine, a metabolite of buprenorphine, or a pharmaceutically acceptable salt of the above, is selected as a therapeutic compound for the treatment of opioid addiction.

Non-limiting examples of opioid receptor ligands suitable for use with the present invention include, oxycodone, hydromorphone, morphine, hydrocodone, fentanyl, oxymorphone, codeine, alfentanil, allylprodine, alphaprodine, anileridine, benzylmorphine, bezitramide, buprenorphine, butorphanol, clonitazene, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, heroin, hydroxypethidine, isomethadone, ketobemidone, levorphanol, levophenacylmorphan, lofentanil, meperidine, meptazinol, metazocine, methadone, metopon, myrophine, narceine, nicomorphine, norlevorphanol, normethadone, nalorphine, nalbuphene, normorphine, norpipanone, opium, oxymorphone, papvereturn, pentazocine, phenadoxone, phenomorphan, phenazocine, phenoperidine, piminodine, piritramide, propheptazine, promedol, properidine, propoxyphene, sufentanyl, tapentadol, tilidine or tramadol, a plurality of mu, kappa, sigma and delta opioid receptors and receptor sub-types as well as their pharmaceutically acceptable salts.

Non-limiting examples of an opioid to which a subject can be addicted to include, oxycodone, hydromorphone, morphine, hydrocodone, fentanyl, oxymorphone, codeine, alfentanil, allylprodine, alphaprodine, anileridine, benzylmorphine, bezitramide, buprenorphine, butorphanol, clonitazene, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, heroin, hydroxypethidine, isomethadone, ketobemidone, levorphanol, levophenacylmorphan, lofentanil, meperidine, meptazinol, metazocine, methadone, metopon, myrophine, narceine, nicomorphine, norlevorphanol, normethadone, nalorphine, nalbuphene, normorphine, norpipanone, opium, oxymorphone, papvereturn, pentazocine, phenadoxone, phenomorphan, phenazocine, phenoperidine, piminodine, piritramide, propheptazine, promedol, properidine, propoxyphene, sufentanyl, tapentadol, tilidine or tramadol, a plurality of mu, kappa, sigma and delta opioid receptors and receptor sub-types as well as their pharmaceutically acceptable salts.

Opioid receptor ligands can be biotransformed and/or metabolized to yield metabolites that are pharmacologically active. A pharmacologically active metabolite can have different physiological effects than a parent compound. For example, norbuprenorphine can be considered to have an analgesic effect that can be 2% of the analgesic effect achieved by buprenorphine in rats. Norbuprenorphine can also be considered to have a respiratory-depressant activity in the rat that is approximately 10 times more potent than the respiratory-depressant activity of buprenorphine.

A pharmacologically active metabolite can have a more potent physiological effect than a parent compound. Certain drugs, such as codeine and tramadol can produce metabolites with pharmacological activity that can be more potent than the parent drugs they derive from, respectively morphine and O-desmethyltramadol. In some embodiments the metabolite can be responsible for the therapeutic action of the parent drug.

A metabolite can be, for example, a substance that is a physiological by-product of a parent compound. Five metabolites of buprenorphine have been identified in rats: 1) buprenorphine-glucuronide; 2) norbuprenorphine; 3) norbuprenorphine-glucuronide; 4) 6-O-desmethylbuprenorphine; and 5) 6-O-desmethylbuprenorphine-glucuronide. In some embodiments, Norbuprenorphine has been identified as a metabolite of buprenorphine that can provide a safe and efficacious treatment of opioid addiction.

The area under the plasma, serum, or blood concentration versus time curve (AUC) can be a useful tool for calculating the relative efficiency of different drug products. The AUC has a number of important uses in toxicology, biopharmaceutics, and pharmacokinetics. The AUC can be used as a measure of drug exposure in toxicology studies. The AUC can be an important parameter in the comparison of drug products in biopharmaceutics. Drug AUC values can be used to determine other pharmacokinetic parameters, such as clearance or bioavailability. Example 4 describes representative AUC's with the device and methods of the invention.

### Polymer Matrix.

The present invention describes a device comprising a therapeutic agent combined with a polymer matrix. A polymer matrix can be an innocuous holder of the therapeutic agent or a polymer matrix can have an active function in determining the dissolution profile of the therapeutic agent. In some embodiments, the polymer is adhesive.

A polymer agent with adhesive properties, such as ethylene vinyl acetate, can be dissolved in an organic solvent and mixed with a therapeutic agent of choice to obtain a homogenous mixture. Such mixture can be used to slowly and steadily release a therapeutic compound in the circulation of a subject. In some embodiments, the polymer matrix of the invention comprises ethylene vinyl acetate. In some embodiments, the therapeutic agent is buprenorphine.

A plurality of polymer matrixes can be used to prepare the disclosed device including, for example, silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, and combinations thereof. In some embodiments the polymer comprises ethylene vinyl acetate.

Additionally, a biodegradable, or non-erodible, polymer can be used in a device of the invention. Such device provides significant advantages over existing devices by deviating the need for subsequent removal. Examples of biodegradable polymers include polyesters such as 3-hydroxypropionate, 3-hydroxybutyrate, 3-hydroxyvalerate, 3-hydroxycaproate, 3-hydroxyheptanoate, 3-hydroxyoctanoate, 3-hydroxynonanoate, 3-hydroxydecanoate, 3-hydroxyundecanoate, 3-hydroxydodecanoate, 4-hydroxybutyrate, 5-hydroxyvalerate, polylactide or polylactic acid including poly(d-lactic acid), poly(1-lactic acid), poly(d,l-lactic acid), polyglycolic acid and polyglycolide, poly(lactic-co-glycolic acid), poly(lactide-co-glycolide), poly(ε-caprolactone) and polydioxanone. Polysaccharides including starch, glycogen, cellulose and chitin can also be used as biodegradable materials.

Further non-erodible, biodegradable materials suitable for inclusion in a device of the invention can include, for example, proteins such as zein, resilin, collagen, gelatin, casein, silk, wool, polyesters, polyorthoesters, polyphosphoesters, polycarbonates, polyanhydrides, polyphosphazenes, polyoxalates, polyaminoacids, polyhydroxyalkanoates, polyethyleneglycol, polyvinylacetate, polyhydroxyacids, polyanhydrides, hydrogels including poly(hydroxyethyl methylacrylate), polyethylene glycol, poly(N-isopropylacrylamide), poly(N-vinyl-2-pyrrolidone), cellulose polyvinyl alcohol, silicone hydrogels, polyacrylamides, and polyacrylic acid. In some embodiments, a biodegradable polymer may be a co-polymer of lactic and glycolic acid.

Furthermore, in order to obtain a sustained-release of buprenorphine, norbuprenorphine or an opioid receptor ligand of choice, a substrate comprising the therapeutically active agent can be coated with a hydrophobic material. Examples of combinations of water insoluble and water soluble materials for the film coat include shellac, polyvinylpyrrolidone, ethyl cellulose, and hydroxypropylmethyl cellulose.

### Dosages.

The device and method of the invention can release a therapeutically effective amount of an opioid receptor ligand, buprenorphine, or a metabolite thereof for an extended period of time after a single administration. A single administration of an opioid receptor ligand, buprenorphine, or a metabolite thereof includes administration of one or more devices or one or more dosage forms at substantially the same time, including for example, a single visit to a physician.

A compound described herein can be present in a device in a range of from about 1 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a device in an amount of about 1 mg, about 5 mg, about 10 mg, about 20 mg, about 25 mg, about 50 mg, about 80 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg. In some embodiments about 80 mg of the compound is present in a device.

The release rate of an opioid receptor ligand, buprenorphine, or a metabolite thereof can be altered by modifying parameters such as the percent drug loading, porosity of the matrix, structure of the implantable device, the hydrophobicity of the matrix, or the number of devices implanted in a human. A hydrophobic coating or a biodegradable coating may be placed over at least a portion of the device to further regulate the rate of release.

In some embodiments, the device or devices can release an opioid receptor ligand, buprenorphine, or a metabolite thereof in-vivo at a rate of about 20 pg/ml per day to about 1 pg/ml per day, about 100 pg/ml per day to about 500 pg/ml per day, about 100 pg/ml per day to about 200 pg/ml per day, about 200 pg/ml per day to about 300 pg/ml per day, about 300 pg/ml per day to about 400 pg/ml per day, or about 400 pg/ml per day to about 500 pg/ml per day. In some embodiments, the ratio of the average to the standard deviation of the amount of an opioid receptor ligand, buprenorphine, or a metabolite thereof released each day may be less than about 1, about 0.5, about 0.3, about 0.2, or about 0.1 for a time period of at least 1 month, at least about 2 months, at least about 3 months, or about 1 months to about 6 months after the device or devices are implanted.

### Device.

An implantable device described herein can be implanted into any mammal, including a human. An implantable device can continuously release therapeutically-effective dosages of an opioid receptor ligand, buprenorphine, or a metabolite of buprenorphine in-vivo over an extended period of time. Implantation of the device can improve compliance with drug dosing regimens and reduce abuse potential. Additionally, a device of the invention can provide a gradual release of the therapeutic agent, providing therapeutically effective plasma levels of an opioid receptor ligand, buprenorphine, or a metabolite of burprenorphine.

Devices can be disk shaped, square or rectangular chip-shaped, cylindrical, square or rectangular rod-shaped. Shapes can be altered by varying the shape of the extruder (Example 1), cutting the extruded material, or by injecting extruded or mixed material into a mold. The shape of the device can be modified according to where the device is implanted, intrinsic tensile-strength of the device, and other factors. In some embodiments, the device is extruded into 26 mm×2.4 mm implants, weighing 125 mg (approximate dimensions, Example 1).

In some embodiments, the device has a length of about 1 cm to about 10 cm, about 1 cm to about 5 cm, about 1 cm to about 2 cm, about 2 cm to about 3 cm, about 3 cm to about 4 cm, or about 4 cm to about 5 cm. In some embodiments, the device may have a mass of about 1 mg to about 10 g, about 10 mg to about 5 g, or about 25 mg to about 1000 mg, about 20 mg to about 50 mg, about 50 mg to about 100 mg, about 100 mg to about 150 mg, about 150 mg to about 200 mg, or about 50 mg to about 200 mg. In some embodiments, the device may have volume of about 0.01 mL to about 2 mL, about 0.05 mL to about 1 mL, about 0.05 mL to about 0.1 mL, about 0.1 mL to about 0.15 mL, about 0.15 mL to about 0.2 mL, about 0.2 mL to about 0.3 mL, or about 0.05 mL to about 0.3 mL.

Multiple implantable devices can be implanted in a subject. The size of the device and the number of devices implanted can depend upon the rate and duration of the sustained release desired. In some embodiments, the number of devices implanted into a human being can be 1, 2, 3, 4, 5, 6, 7, 8, 9, 10, 11, 12, 13, 14, or 15. In some embodiments, the total weight of all devices implanted is about 80 mg to about 320 g.

For delivery of an opioid receptor ligand, buprenorphine, or a metabolite of burprenorphine to a human being for about 24 weeks, 1, 2 or 4 devices can be implanted, wherein the total weight of the devices can be about 80 mg to about 320 mg. For delivery of opioid receptor ligand, buprenorphine, or a metabolite of burprenorphine to a human being for about 52 weeks, 1, 2, 3, or 4 devices can be implanted, wherein the total weight of the devices may be about 80 mg to about 320 mg. For delivery of an opioid receptor ligand, buprenorphine, or a metabolite of burprenorphine to a human being for about 18 months, 1, 2, 3, 4, 5, or 6 devices can be implanted, wherein the total weight of the devices may be about 80 mg to about 480 mg. For delivery of opioid receptor ligand, buprenorphine, or a metabolite of burprenorphine to a human being for about 24 months, 1, 2, 3, 4, 5, 6, 7, or 8 devices may be implanted, wherein the total weight of the devices may be about 80 mg to about 640 mg.

An implantable device can be administered by implantation in an individual, and an implantable device can be administered by a physician, a nurse, a nurse practitioner, and a plurality of health care provider. The device can be implanted subcutaneously in any of a variety of sites of the body, such as the upper arm, the back, the abdomen. Multiple implantable devices can be administered, and multiple implantable devices can be administered to different body sites.

A device can have a burst period. A burst period can be a time period of substantially constant release. A burst period can occur about 1 hour, about 2 hours, about 6 hours, about 12 hours, about 24 hours, about 36 hours, about 48 hours, about 60 hours, and about 72 hours after implantation. A burst period can be reduced by washing the device prior to implantation with, for example, an alcohol.

### Pharmaceutically Acceptable Salts.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, a iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### EXAMPLES

### Example 1

### A Device Providing an Implantable Formulation to Treat Opioid Addiction

The device of the invention comprises an implantable polymeric matrix and an active compound for the treatment of opioid addiction. This example describes an embodiment wherein the polymeric matrix is ethylene-vinyl acetate (EVA) copolymer and the active compound is Buprenorphine Hydrochloride, extruded into 26 mm×2.4 mm implants, weighing 125 mg (approximate dimensions).

Materials and Methods. Reagents: a) milled ethylene vinyl acetate copolymer (EVA, 33% VA) (600 μm), supplied by Southwest Research Institute; b) buprenorphine hydrochloride USP <53 μm; c) buprenorphine hydrochloride USP 53-180 μm, supplied by Sigma-Aldrich™ (sieved at SwRI); c) buprenorphine hydrochloride USP 53-180 μm, supplied by Diosynth™ (sieved at SwRI); d) ethylene vinyl acetate copolymer (EVA, 33% VA) Sigma Aldrich; e) alcohol USP, supplied by Equistar; and f) pre-blended EVA/buprenorphine HCl.

Materials and Methods. Equipment: a) Patterson-Kelley, blend master lab blender, yoke blender, twin shell, 1-quart; b) thermo-electron twin screw extruder 16 TC 25:1 TC; c) tapered 2.40 mm Die TI-01-0804-001; d) Die TI-01-0804-005 2.50 mm (horizontal feed); e) brabender volumetric single screw feeder DSR 28; f) beta laser mike/accuscan 3, model#LD1010XY-S; g) analytical balance, AT261 delta range mettle Toledo, capable of 0.0001 g precision; h) mitutoyo digimatic caliper, CD-6″ C; i) model 610 cold air gun, pelmar Engineering Ltd.; j) waters 2690/95-2996 PDA Detector (HPLC); k) gas chromatography instrument (6850 agilent); 1) mettle DL 18, Karl Fisher water determination instrument; m) DiSTek™ 2100B and 2100C, dissolution apparatus; n) globepharma unit dose sampling thief (sample die 0.25 CC) model I; o) KVB micro sampler SS, 12 with a 0.2 CC head; p) Trol-Mation conveyor model: BC-0.75x24-US425-401U-4GN180-RAA; q) VWR Forced air oven model: 1350 FMS; r) VEW vacuum oven model: 1450 MS; and s) 3M ionizing air gun model 980.

Implants comprising particulates of buprenorphine and EVA were produced via hot melt extrusion utilizing a twin co-rotating screw extruder. The implants were washed in 95% (v/v) ethanol to remove surface Buprenorphine HCl to control the initial “burst” release of drug upon implantation. Development of the device focused on four distinct stages: a) the active ingredient and polymeric carrier were uniformly blended; b) the blended mixture was extruded and cut into implants of uniform weight and diameter; and c) the implants were washed to remove excess active ingredient from the surface, and the washed implants were dried to remove residual ethanol (from washing).

TABLE 1 illustrates the uniformity of a particulate of buprenorphine and EVA obtained under three different blending conditions. The EVA copolymer and Buprenorphine HCl were added to the pre-blended material and tumbled at 25 rpm for 30 minutes.

TABLE 1
(% w/w Buprenorphine HCl)

| | 5 minute blend | 30 minute blend | 60 minute blend |
|---|---|---|---|
| Mean | 78.42 | 77.84 | 75.27 |
| STD Dev | 2.5 | 3.6 | 2.5 |
| RSD | 3 | 5 | 3 |
| Min | 75.69 | 72.36 | 71.63 |
| Max | 80.52 | 81.82 | 78.72 |

TABLE 2 illustrates average particle sizes of particulates of EVA and Buprenorphine HCl obtained under three different conditions.

TABLE 2

| | Specifications | Results |
|---|---|---|
| Particle Size | D(0.1) | 1—10.651 μm |
| | | 2—10.271 μm |
| | | 3—10.265 μm |
| | | Avg: 10.396 μm |
| Particle Size | D(0.5) | 1—94.639 μm |
| | | 2—91.762 μm |
| | | 3—90.847 μm |
| | | Avg: 92.416 μm |
| Particle Size | D(0.9) | 1—303.249 μm |
| | | 2—296.419 μm |
| | | 3—293.166 μm |
| | | Avg: 297.611 μm |

Devices were further evaluated based on appearance, rod diameter, rod length, rod weight, tensile strength, and dissolution parameters described in TABLE 3.

TABLE 3

| Test | Specifications | |
|---|---|---|
| Appearance | | |
| Rod Diameter | 2.4 +/− 0.24 | mm |
| Rod Length | 26 +/− 2.6 | mm |
| Rod Weight | 125 +/− 12.5 | mg |

| | |
|---|---|
| Tensile Strength | Record Results ranging from: |
| | Ave = 65678 g/cm² to |
| | Ave = 102721 g/cm² |

| | Record Results ranging from: | | |
|---|---|---|---|
| | Hours | % Dissolved Sample 1 | % Dissolved Sample 2 |
| Dissolution | 4 | 1.9 | 3.5 |
| | 24 | 8.4 | 15.8 |
| | 48 | 15.6 | 26.3 |
| | 120 | 29.2 | 42.1 |
| | 168 | 35.7 | 46.5 |

The process described herein provided a particulate of EVA and Buprenorphine HCl that when extruded into devices of approximately 26 mm×2.4 mm in size, weighing approximately 125 mg, and implanted into subjects, provided the blood plasma profiles described in Example 2.

### Example 2

### Pharmacokinetic Parameters of Implants for the Treatment of Opioid Addiction in Subjects with Opiate Dependence

Buprenorphine implants were administered to subjects for treatment of opioid dependence. The pharmacokinetics and effectiveness of the implants for the treatment of opiate addiction are illustrated in the following example.

The study was designed as an open-label, sequential dose-group study of 12 patients (6 patients per dose group) with DSM-IV defined opioid dependence who were in a maintenance treatment program with sublingual buprenorphine. Patients were switched from a sub-lingual buprenorphine therapy to treatment with devices of the invention. For the 2 implant dose group, patients maintained on sub-lingual buprenorphine 8 mg (1 tablet) daily were switched to 2 device implants places subcutaneously in one arm for 6 months. Rescue therapy with sublingual buprenorphine was provided to patients who exhibited inadequate therapeutic control as indicated by their clinical condition.

Prior to insertion of implants and while patients were receiving sublingual maintenance doses, samples for determination of buprenorphine and norbuprenorphine concentrations were obtained at 1 hour post dose representing approximate peak concentrations, and at 24 hours after the previous dose.

Plasma samples for determination of buprenorphine and norbuprenorphine concentration were obtained at 0, 3, 6, 9, 12, 16, 20, 24, 30, 36, and 48 hours; Days 3, 4, 5, 6, 7, 10, 14, and 21; Weeks 4, 6, 8, 10, 12, 14, 16, 18, 20, 22, and 24 versus time of insertion of implants.

FIG. 1 illustrates the mean plasma buprenorphine concentration with the device and methods of the invention during twenty four weeks of study. FIG. 2 illustrates the comparison of the mean plasma buprenorphine concentration with the device and methods of the invention on an open labeled study and a double blind study.

During treatment with a device(s) of the invention, buprenorphine and norbuprenorphine changes in the observed plasma concentration over time curves were measured. The release rate of buprenorphine initially comprised a time period of substantial constant release, followed by a plateau in the release rate of buprenorphine from the implants.

The time course of buprenorphine concentrations for a device(s) of the invention was consistent across patients with an initial increase over the first 24 hours after insertion and a multi-phase decrease thereafter. The plateau phase was reached within 21 days in all patients and no patient had a reduction in buprenorphine concentrations by more than 50% from Day 17 to removal. Groups with higher doses, or a higher number of implanted devices, were characterized by an overall tendency of a more rapid achievement of the plateau phase of drug release.

TABLE 4 shows the summary of buprenorphine pharmacokinetic parameters by patient and dosing group (2 or 4 implants) with summary statistics. The following variables for buprenorphine were determined from the observed data over the time period of device insertion: 1) Cₘₐₓ, maximum concentration; 2) tₘₐₓ, time of maximum concentration; 3) Cₘᵢₙ, minimum concentration; 4) Cₘₐₓ/Cₘᵢₙ, ratio maximum/minimum concentration; 5) t_{1/2} half-life from Day 21 to the last observation before removal; and 6) Cₐᵥₑ, average concentration from Day 21 to the last observation before removal calculated as AUC/time.

TABLE 4

| | | | | | | Cₐᵥₑ | t_{1/2} | |
|---|---|---|---|---|---|---|---|---|
| | | | | | | Day 21 | Day 21 | C_{1/2} |
| | | | | | | to | to | After |
| | | Cₘₐₓ | tₘₐₓ | Cₘᵢₙ | | removal | removal | removal |
| Patient | Implants | (pg/ml) | (H) | (pg/ml) | Cₘₐₓ/Cₘᵢₙ | (pg/ml) | (weeks) | (H) |
| 001-001 | 2 | 2630 | 9.05 | 282 | 9.33 | 366 | 90.2 | 30.0 |
| 001-002 | 2 | 1980 | 16.00 | 237 | 8.35 | 344 | 25.1 | 32.0 |
| 001-003 | 2 | 2040 | 19.08 | 262 | 7.79 | 363 | 50.3 | NE |
| 002-001 | 2 | 1490 | 23.83 | 305 | 4.89 | 435 | 25.0 | NE |
| 002-002 | 2 | 1630 | 20.08 | 213 | 7.65 | 253 | NE | 14.4 |
| 003-003 | 2 | 2200 | 15.97 | 378 | 5.82 | 446 | NE | 18.6 |
| Mean | | 1995 | 17.34 | 279.5 | 7.31 | 367.8 | 47.7 | 23.8 |
| SD | | 409 | 5.00 | 58.1 | 1.65 | 69.9 | 30.8 | 8.6 |
| Median | | 2010 | 17.54 | 272.0 | 7.72 | 364.5 | 37.7 | 24.3 |
| 001-005 | 4 | | | | | 886 | 47.8 | NE |
| 001-006 | 4 | 3460 | 12.0 | 517 | 6.69 | 703 | 83.5 | 15.6 |
| 001-007 | 4 | 3021 | 16.0 | 567 | 5.33 | 690 | NE | 13.1 |
| 001-008 | 4 | 2740 | 15.75 | 476 | 5.76 | 547 | 95.9 | 15.8 |
| 002-003 | 4 | 2970 | 12.0 | 593 | 5.01 | 758 | NE | 9.7 |
| 003-004 | 4 | 3960 | 24.0 | 509 | 7.78 | 710 | 31.6 | 14.3 |
| Mean | | 3230 | 15.95 | 532.4 | 6.11 | 715.7 | 64.7 | 13.7 |
| SD | | 484 | 4.90 | 47.0 | 1.13 | 109.6 | 30.0 | 2.5 |
| Median | | 3020 | 15.75 | 517.0 | 5.76 | 706.5 | 65.7 | 14.3 |

The half-life for buprenorphine concentrations from Day 21 to removal could not be estimated in 4 patients as their profiles were practically flat over this time period. The shortest half-life during the plateau phase was 25 weeks. Cₘₐₓ was on average 62% higher in the higher dose group as compared with the lower dose group, whereas Cave from Day 21 to removal was 93% higher, a close to proportional increase with dose. The buprenorphine terminal half-life after device removal was estimated in 9 of the patients.

TABLE 5 shows the summary of norbuprenorphine pharmacokinetic parameters by patient and dosing group (2 or 4 implants) with summary statistics. The following variables for norbuprenorphine were determined from the observed data over the time period of device insertion: 1) Cₐᵥₑ, average concentration from Day 21 to the last observation before removal calculated as AUC/time; 2) t_{1/2} half-life from Day 21 to the last observation before removal.

TABLE 5

| | | Cₐᵥₑ | t_{1/2} |
|---|---|---|---|
| | | Day 21 | Day 21 |
| | | to | to |
| | | removal | removal |
| Patient | Implants | (pg/ml) | (weeks) |
| 001-001 | 2 | 76.8 | 22.4 |
| 001-002 | 2 | 239.1 | 401.3 |
| 001-003 | 2 | 109.9 | 202.4 |
| 002-001 | 2 | 104.1 | 13.3 |
| 002-002 | 2 | 100.0 | NE |
| 003-003 | 2 | 103.8 | NE |
| Mean | | 122.3 | 159.9 |
| SD | | 58.4 | 183.0 |
| Median | | 104.0 | 112.4 |
| 001-005 | 4 | 390.9 | NE |
| 001-006 | 4 | 344.2 | 145.8 |
| 001-007 | 4 | 238.5 | NE |
| 001-008 | 4 | 441.3 | NE |
| 002-003 | 4 | 303.8 | NE |
| 003-004 | 4 | 231.2 | NE |
| Mean | | 324.8 | |
| SD | | 83.5 | |
| Median | | 324.0 | |

### Example 3

### Delivery of Buprenorphine Via an Implantable Delivery System In Vivo: Clinical Report of a Randomized, Placebo and Active-Controlled, Multi-Center Study of Patients with Opioid Dependence Treated with Devices of the Invention

The efficacy and superiority of the device and methods of the invention versus placebo and previously available therapies in adult subjects with Diagnostic and Statistical Manual of Mental Disorders IV text revision (DSM-IV-TR)-defined opioid dependence over Weeks 1 through 24 of outpatient treatment through the assessment of thrice-weekly urine toxicology results and illicit drug self-reported data were evaluated.

This was a randomized, placebo- and active-controlled, multicenter study of the device and methods of the invention in adult patients with opioid dependence. The following groups were evaluated: Group A (4 devices implanted, blinded); Group B (4 placebo implants, blinded); Group C (12 to 16 mg once daily of sub-lingual buprenorphine). FIG. 3 illustrates a schematic of the treatment protocol used in two open label treatment studies. The implant visit occurred within 14 days of the stat of induction. FIG. 4 illustrates a representative site of implantation of a device of the invention in a human. FIG. 5 illustrates implantation of a device in the subdermal tissue.

For groups A and B, implants were inserted in the subject's inner upper arm in a brief, in-office procedure, by implant-procedure certified clinicians. FIG. 6 illustrates the specifications of the applicator used to insert a device of the invention in a subject. The same procedure was performed for implant dose increases. At the end of treatment or at early discontinuation, implants were removed in a brief, in-office procedure by the same certified clinicians. All subjects attended twice-weekly manual guided drug counseling during study Weeks 1 through 12 and weekly drug counseling during Weeks 13 through 24 with the ability to attend additional sessions within protocol-specified limits. All subjects were eligible for receiving limited supplemental SL BPN if they met pre-specified criteria for withdrawal symptoms and cravings or at the investigators discretion. Enrolled subjects were male and female, age 18 to 65 years old who met DSM-IV-TR criteria for current opioid dependence.

The primary analysis was a comparison of the cumulative distribution of the percentage of urine samples negative at Week 24 (Weeks 1 through 24) in the 2 treatment groups by using an exact stratified Wilcoxon rank sum (van Elteren) test with (pooled) site and gender as stratification variables. There was a statistically significant difference (P<0.0001) between the device and methods of the invention and placebo treatments for the probability of urine samples negative for illicit opioids from Weeks 1 through 24. There was also a statistically significant difference (P<0.0001) between the device and methods of the invention and placebo in favor of the device and methods of the invention for the probability of urine samples negative for illicit opioids from Weeks 1 through 24 with imputation based on illicit drug self-report data. There was a statistically significant difference (P<0.0001) between the device and methods of the invention and placebo in favor of the device and methods of the invention for the probability of urine samples negative for illicit opioids from Weeks 1 through 16 with weeks 17 through 24, with a higher probability of negative urine samples in the group comprising the device and methods of the invention. At the end of treatment or at early discontinuation, implants were removed in a brief, in-office procedure.

### Example 4

### Delivery of Buprenorphine Via an Implantable Delivery System In Vivo: Buprenorphine and Norbuprenorphine Blood Plasma Profile in Subjects without Rescue Medication

Buprenorphine implants were administered to subjects for treatment of opioid dependence. The efficacy results and a tabulation of individual subject data and secondary efficacy analysis for the treatment of opiate addiction are illustrated in the following example.

Buprenorphine concentration data for 61 subjects were measured. FIG. 7 and FIG. 8 display the individual (plotted as individual data points) and the weekly mean (trendline between individual data points) concentrations for buprenorphine and norbuprenorphine on a semi-log scale for subjects with 4 implants who did not receive supplemental buprenorphine. Individual concentrations at Week 0 are prior to implantation and at 12 to 24 hours are after the most recent sublingual buprenorphine treatment.

### Example 5

### Half-Life, Renal Excretion, and Area Under the Plasma Concentration Time Curve (AUC) of Buprenorphine in the Blood Plasma of Subjects with Opiate Dependence Treated with Devices and Methods of the Invention

Buprenorphine implants were administered to subjects for treatment of opioid dependence. The pharmacokinetics and effectiveness of the implants for the treatment of opiate addiction are illustrated in the following example.

The study was designed as an open-label, sequential dose-group study of 12 patients (6 patients per dose group) with DSM-IV defined opioid dependence who were in a maintenance treatment program with sublingual buprenorphine. Patients were switched from a sub-lingual buprenorphine therapy to treatment with devices of the invention. For the 2 implant dose group, patients maintained on sub-lingual buprenorphine 8 mg (1 tablet) daily were switched to 2 device implants places subcutaneously in one arm for 6 months. Rescue therapy with sublingual buprenorphine was provided to patients who exhibited inadequate therapeutic control as indicated by their clinical condition.

Prior to insertion of implants and while patients were receiving sublingual maintenance doses, samples for determination of buprenorphine and norbuprenorphine concentrations were obtained at 1 hour post dose representing approximate peak concentrations, and at 24 hours after the previous dose.

Plasma samples for determination of buprenorphine and norbuprenorphine concentration were obtained at 0, 3, 6, 9, 12, 16, 20, 24, 30, 36, and 48 hours; Days 3, 4, 5, 6, 7, 10, 14, and 21; Weeks 4, 6, 8, 10, 12, 14, 16, 18, 20, 22, and 24 versus time of insertion of implants. Additional samples were obtained at 10 and 30 minutes; 1, 2, 4, 6, 9, 12, 24, 36, and 48 hours after device removal.

The exponential decay of the plateau pattern of release of buprenorphine from a device(s) of the invention was measured. The biological half-lives “alpha half-life” (α-Half Life, hours) and “beta half-life” (β-Half Life, hours) of buprenorphine measured patterns of drug distribution and elimination from plasma circulation. The “terminal half-life” (γ-Half life, weeks) of buprenorphine was measured patterns of drug elimination from the system and is described in Table 6.

TABLE 6

| | | | | | | Time from |
|---|---|---|---|---|---|---|
| | | | | | Model | implant to |
| | | | | | predicted | reaching 2x |
| | | α-Half | β-Half | γ-Half | concentration | last |
| | | Life | Life | life | at removal | concentration |
| Patient | Implants | (hours) | (hours) | (weeks) | (pg/ml) | (h) |
| 001-001 | 2 | 9.41 | 109 | 120 | 338 | 203 |
| 001-002 | 2 | 7.55 | 124 | 29.9 | 263 | 396 |
| 001-003 | 2 | 4.17 | 206 | 457 | 342 | 259 |
| 002-001 | 2 | 115^{b} | 3578^{bc} | 35.7 | 342 | 313 |
| 002-002 | 2 | 39.7 | 174 | 131,709^{c} | 252 | 180 |
| 003-003 | 2 | 2.94 | 52.3 | 350,207^{c} | 447 | 101 |
| Mean | | 12.75 | 133.1 | 160.7 | 330.7 | 242.0 |
| SD | | 15.28 | 59.54 | 201.8 | 70.2 | 104.2 |
| Median | | 7.55 | 124.0 | 77.9 | 340.0 | 231.0 |
| 001-006 | 4 | 3460 | 12.0 | 517 | 6.69 | 703 |
| 001-007 | 4 | 3021 | 16.0 | 567 | 5.33 | 690 |
| 001-008 | 4 | 2740 | 15.75 | 476 | 5.76 | 547 |
| 002-003 | 4 | 2970 | 12.0 | 593 | 5.01 | 758 |
| 003-004 | 4 | 3960 | 24.0 | 509 | 7.78 | 710 |
| Mean | | 3230 | 15.95 | 532.4 | 6.11 | 715.7 |
| SD | | 484 | 4.90 | 47.0 | 1.13 | 109.6 |
| Median | | 3020 | 15.75 | 517.0 | 5.76 | 706.5 |

^{a}Calculated for Day 154 for patient 2-1 due to erroneous data at later time points.
^{b}Not included in the summary statistics due to suboptimal model for the initial phase.
^{c}Not included in the summary statistics due to poor estimate with CV % >3000.

The amount of buprenorphine excreted in the urine (ng), the buprenorphine rate of plasma AUC (pg/mL*h), and the rate of renal clearance were measured and are described in Table 7.

TABLE 7

| | | | Amount | | |
|---|---|---|---|---|---|
| | | | excreted | Plasma | Renal |
| | | | in urine | AUC | clearance |
| Patient | Implants | Hours | (ng) | (pg/mL * h) | (mL/min) |
| 001-001 | 2 | 0-24 | 427 | 45,819 | 0.0065 |
| | | 24-48 | NS | | |
| 001-002 | 2 | 0-24 | 2015 | 36,285 | 0.0386 |
| | | 24-48 | 759 | 32,760 | 0.0161 |
| 001-003 | 2 | 0-24 | 627 | 35,917 | 0.0121 |
| | | 24-48 | NS | | |
| 002-001 | 2 | 0-24 | 1528 | 19,591 | 0.0552 |
| | | 24-48 | 1643 | 31,770 | 0.0359 |
| 002-002 | 2 | 0-24 | 1998 | 21,948 | 0.0632 |
| | | 24-48 | 5031 | 30,840 | 0.1133 |
| 003-003 | 2 | 0-24 | 1706 | 41,836 | 0.0283 |
| | | 24-48 | 2618 | 36,510 | 0.0498 |
| Mean | | | | | 0.0418 |
| SD | | | | | 0.0313 |
| Median | | | | | 0.0373 |
| 001-005 | 4 | 0-24 | 583 | NC | NC |
| | | 24-48 | 894 | NC | NC |
| 001-006 | 4 | 0-24 | 1863 | 58,490 | 0.0221 |
| | | 24-48 | 1266 | 45,240 | 0.0194 |
| 001-007 | 4 | 0-24 | 1030 | 49,165 | 0.0146 |
| | | 24-48 | 1058 | 35,670 | 0.0206 |
| 001-008 | 4 | 0-24 | 560 | 44,431 | 0.0088 |
| | | 24-48 | 6304 | 40,410 | 0.1083 |
| 002-003 | 4 | 0-24 | 1699 | 48,998 | 0.0241 |
| | | 24-48 | 2490 | 49,800 | 0.0347 |
| 003-004 | 4 | 0-24 | 4310 | 68,715 | 0.0436 |
| | | 24-48 | 3906 | 72,000 | 0.0377 |
| Mean | | | | | 0.0334 |
| SD | | | | | 0.0284 |
| Median | | | | | 0.0231 |

NS: no sample.
NC: Not calculated.

The amount of norbuprenorphine excreted in the urine (ng), the norbuprenorphine rate of plasma AUC (pg/mL*h), and the rate of renal clearance were measured and are described in Table 7.

TABLE 8

| | | | Amount | | |
|---|---|---|---|---|---|
| | | | excreted | Plasma | Renal |
| | | | in urine | AUC | clearance |
| Patient | Implants | Hours | (ng) | (pg/mL * h) | (mL/min) |
| 001-001 | 2 | 0-24 | 37,290 | 24,379 | 1.062 |
| | | 24-48 | NS | | |
| 001-002 | 2 | 0-24 | 105,400 | 49,780 | 1.470 |
| | | 24-48 | 35,640 | 46,200 | 0.536 |
| 001-003 | 2 | 0-24 | 59,695 | 24,287 | 1.707 |
| | | 24-48 | NS | | |
| 002-001 | 2 | 0-24 | 105,000 | 27,032 | 2.697 |
| | | 24-48 | 89,900 | 24,618 | 2.536 |
| 002-002 | 2 | 0-24 | 135,900 | 24,987 | 3.777 |
| | | 24-48 | 77,415 | 25,638 | 2.097 |
| 003-003 | 2 | 0-24 | 65,142 | 18,773 | 2.410 |
| | | 24-48 | 63,840 | 12,846 | 3.451 |
| Mean | | | | | 2.174 |
| SD | | | | | 1.017 |
| Median | | | | | 2.254 |
| 001-005 | 4 | 0-24 | 32,780 | NC | NC |
| | | 24-48 | 35,300 | NC | NC |
| 001-006 | 4 | 0-24 | 163,750 | 87,440 | 1.300 |
| | | 24-48 | 146,250 | 76,920 | 1.320 |
| 001-007 | 4 | 0-24 | 59,186 | 85,055 | 0.483 |
| | | 24-48 | 55,440 | 60,930 | 0.632 |
| 001-008 | 4 | 0-24 | 18,900 | 74,000 | 0.177 |
| | | 24-48 | 235,200 | 70,290 | 2.324 |
| 002-003 | 4 | 0-24 | 56,640 | 41,040 | 0.958 |
| | | 24-48 | 71,900 | 29,250 | 1.707 |
| 003-004 | 4 | 0-24 | 128,370 | 23,182 | 3.846 |
| | | 24-48 | 115,520 | 22,989 | 3.490 |
| Mean | | | | | 0.0334 |
| SD | | | | | 0.0284 |
| Median | | | | | 0.0231 |

NS: no sample.
NC: Not calculated.

### EMBODIMENTS

The following non-limiting embodiments provide illustrative examples of the invention, but do not limit the scope of the invention.

### Embodiment 1

A method of treating opioid addiction in a subject in need of relief thereof, the method comprising: implanting a device comprising a particulate of at least one opioid receptor ligand, and a polymer matrix, to the subject, wherein the device is selected based on a therapeutic effect of the opioid receptor ligand, and wherein the device releases a therapeutically-effective amount of the opioid receptor ligand.

### Embodiment 2

The method of Embodiment 1, wherein the opioid receptor ligand is an opiate.

### Embodiment 3

The method of any one of Embodiments 1 and 2, wherein the opioid receptor ligand is a synthetic opioid.

### Embodiment 4

The method of any one of Embodiments 1-2, wherein the opioid receptor ligand is a semi-synthetic opioid.

### Embodiment 5

The method of any one of Embodiments 1-4, wherein the opioid receptor ligand is a partial opioid agonist.

### Embodiment 6

The method of any one of Embodiment 1-5, wherein the opioid receptor ligand is buprenorphine.

### Embodiment 7

The method of any one of Embodiments 1-5, wherein the opioid receptor ligand is a metabolite of buprenorphine.

### Embodiment 8

The method of Embodiment 7, wherein the metabolite is norbuprenorphine.

### Embodiment 9

The method of any one of Embodiments 1-8, wherein the particulate is in an amorphous state.

### Embodiment 10

The method of any one of Embodiments 1-8, wherein the particulate is in a solid state.

### Embodiment 11

The method of any one of Embodiments 1-6, 9, and 10, wherein the device comprises particles of buprenorphine or a pharmaceutically acceptable salt thereof ranging from about 5 μm to about 600 μm in size.

### Embodiment 12

The method of any one of Embodiments 1-6, 9, and 10, wherein the device comprises particles of buprenorphine or a pharmaceutically acceptable salt thereof having an average particle size from about 50 μm to about 180 μm in size.

### Embodiment 13

The method of any one of Embodiments 1-6, 9, and 10, wherein the device comprises particles of buprenorphine of about 10 μm in size.

### Embodiment 14

The method of any one of Embodiments 1-13, wherein the opioid receptor ligand is a salt.

### Embodiment 15

The method of any one of Embodiments 1-6, wherein the particulate is associated with a dissolution profile.

### Embodiment 16

The method of Embodiment 15, wherein the dissolution profile correlates to a plasma concentration of a therapeutically-effective amount of buprenorphine or a pharmaceutically-acceptable salt thereof.

### Embodiment 17

The method of Embodiment 15, wherein the dissolution profile correlates to a plasma concentration of the opioid receptor ligand from about 100 pg/ml to about 900 pg/ml.

### Embodiment 18

The method of Embodiment 15, wherein the dissolution profile correlates to a plasma concentration of the opioid receptor ligand from about 200 pg/ml to about 4,500 pg/ml.

### Embodiment 19

The method of Embodiment 15, wherein the dissolution profile correlates to a plasma concentration of a metabolite of the opioid receptor ligand from about 20 pg/ml to about 500 pg/ml.

### Embodiment 20

The method of Embodiment 19, wherein the metabolite is norbuprenorphine.

### Embodiment 21

A method of treating opioid addiction in a subject in need of relief thereof, the method comprising administering to the subject a device comprising buprenorphine, and a polymer matrix, wherein the device comprises a tensile strength in a range of about 10,000 g/cm² to about 110,000 g/cm²; wherein the administering comprises implanting the device into the subject; and wherein the device releases a therapeutically-effective amount of buprenorphine, or the pharmaceutically-acceptable salt thereof, to the subject.

### Embodiment 22

The method of Embodiment 21, wherein the tensile strength of the device ranges from about 10,000 g/cm² to about 110,000 g/cm².

### Embodiment 23

The method of any one of Embodiments 21-22, wherein the tensile strength of the device has an average from about 45,000 g/cm² to about 80,000 g/cm².

### Embodiment 24

The method of any one of Embodiments 21-23, wherein the tensile strength of the device ranges from about 75,000 g/cm² to about 110,000 g/cm².

### Embodiment 25

The method of any one of Embodiments 21-24, wherein the device comprises particles of buprenorphine ranging from about 5 μm to about 600 μm in size.

### Embodiment 26

The method of any one of Embodiments 21-25, wherein the device comprises particles of buprenorphine having an average particle size from about 50 μm to about 180 μm in size.

### Embodiment 27

The method of any one of Embodiments 21-26, wherein the device comprises particles of buprenorphine of about 10 μm in size.

### Embodiment 28

The method of any one of Embodiments 21-27, wherein the device is selected based on the tensile strength of the device.

### Embodiment 29

The method of Embodiment 28, wherein the selecting the device comprises selecting a device that releases particles of buprenorphine, wherein the particles are associated with a dissolution profile.

### Embodiment 30

The method of Embodiment 29, wherein the buprenorphine is a salt.

### Embodiment 31

The method of Embodiment 29, wherein the dissolution profile correlates to a plasma concentration of buprenorphine from about 100 pg/ml to about 900 pg/ml.

### Embodiment 32

The method of Embodiment 29, wherein the dissolution profile correlates to a plasma concentration of buprenorphine from about 200 pg/ml to about 4,500 pg/ml.

### Embodiment 33

The method of Embodiment 29, wherein the dissolution profile correlates to a plasma concentration of a metabolite of buprenorphine from about 20 pg/ml to about 500 pg/ml.

### Embodiment 34

The method of Embodiment 33, wherein the metabolite is norbuprenorphine.

### Embodiment 35

An implantable device comprising: buprenorphine and a polymer matrix,

wherein the implantable device has a tensile strength in a range of about 10,000 g/cm² to about 110,000 g/cm², wherein upon implantation in a human the implant releases a therapeutically-effective amount of buprenorphine to the human.

### Embodiment 36

The device of Embodiment 35, wherein the polymer matrix is adhesive.

### Embodiment 37

The device of any one of Embodiments 35 and 36, wherein the polymer matrix is non-erodible.

### Embodiment 38

The device of any one of Embodiments 35-37, wherein the device releases at least one particle comprising buprenorphine.

### Embodiment 39

The device of any one of Embodiments 35-38, wherein the particle is in an amorphous state.

### Embodiment 40

The device of any one of Embodiments 35-39, wherein the particle is in a solid state.

### Embodiment 41

The device of any one of Embodiments 35-40, wherein the polymer comprises ethylene vinyl acetate.

### Embodiment 42

The device of any one of Embodiments 35-41, wherein the device releases a therapeutically effective level of buprenorphine for at least 24 weeks.

### Embodiment 43

The device of any one of Embodiments 35-42, wherein the device releases particles of buprenorphine, wherein the particles are associated with a dissolution profile.

### Embodiment 44

The device of Embodiments 43, wherein the buprenorphine is buprenorphine hydrochloride.

### Embodiment 45

The device of Embodiment 43, wherein the dissolution profile correlates to a plasma concentration of buprenorphine from about 100 pg/ml to about 900 pg/ml.

### Embodiment 46

The device of Embodiment 43, wherein the dissolution profile correlates to a plasma concentration of buprenorphine from about 200 pg/ml to about 4,500 pg/ml.

### Embodiment 47

The device of Embodiment 43, wherein the dissolution profile correlates to a plasma concentration of a metabolite of buprenorphine from about 20 pg/ml to about 500 pg/ml.

### Embodiment 48

The device of Embodiment 47, wherein the metabolite is norbuprenorphine.

### Embodiment 49

A method of treating opioid addiction in a subject in need of relief thereof, the method comprising: implanting a device comprising buprenorphine and a polymer matrix to the subject, wherein the device provides a plasma concentration of a metabolite of buprenorphine of about 50 pg/ml to about 3,000 pg/ml over a period of at least about 24 weeks.

### Embodiment 50

The method of Embodiment 49, wherein the plasma concentration of the metabolite of buprenorphine ranges from about 1,000 pg/ml to about 3,000 pg/ml.

### Embodiment 51

The method of Embodiment 49, wherein the plasma concentration of the metabolite of buprenorphine ranges from about 50 pg/ml to about 600 pg/ml.

### Embodiment 52

The method of Embodiment 49, wherein the plasma concentration of the metabolite of buprenorphine ranges from about 500 pg/ml to about 1,000 pg/ml.

### Embodiment 53

The method of Embodiment 49, wherein the plasma concentration of the metabolite of buprenorphine ranges from about 300 pg/ml to about 900 pg/ml.

### Embodiment 54

The method of Embodiment 49, wherein the metabolite of buprenorphine is a pharmaceutically-acceptable salt.

### Embodiment 55

The method of any one of Embodiments 49-54, wherein the metabolite is norbuprenorphine.

What is claimed is:

1. A method of treating hydrocodone addiction in a subject in need of relief thereof, the method comprising implanting into a subdermal tissue of the subject a device comprising at least one opioid receptor ligand and a polymer matrix, and subsequently sublingually administering the opioid receptor ligand to the subject, wherein the device releases a therapeutically-effective amount of the opioid receptor ligand.

2. The method of claim 1, wherein the opioid receptor ligand is an opiate.

3. The method of claim 1, wherein the opioid receptor ligand is a synthetic opioid.

4. The method of claim 1, wherein the opioid receptor ligand is a semi-synthetic opioid.

5. The method of claim 1, wherein the opioid receptor ligand is a partial opioid agonist.

6. The method of claim 1, wherein the opioid receptor ligand is buprenorphine.

7. The method of claim 6, wherein the buprenorphine is buprenorphine hydrochloride.

8. The method of claim 1, wherein the opioid receptor ligand is a metabolite of buprenorphine.

9. The method of claim 7, wherein the metabolite is norbuprenorphine.

10. The method of claim 1, further comprising sublingually administering an opioid therapy to the subject prior to implantation.

11. The method of claim 1, wherein the opioid receptor ligand is buprenorphine in particles ranging from about 5 μm to about 600 μm in size.

12. The method of claim 1, wherein the opioid receptor ligand is buprenorphine in particles having an average particle size from about 50 μm to about 180 μm in size.

13. The method of claim 1, wherein the opioid receptor ligand is buprenorphine in particles having an average particle size of about 10 μm.

14. The method of claim 1, wherein the opioid receptor ligand is a salt.

15. The method of claim 1, wherein the device is associated with a dissolution profile.

16. The method of claim 15, wherein the dissolution profile correlates to a plasma concentration of a therapeutically-effective amount of buprenorphine.

17. The method of claim 15, wherein the dissolution profile correlates to a plasma concentration of the opioid receptor ligand from about 100 pg/ml to about 900 pg/ml.

18. The method of claim 15, wherein the dissolution profile correlates to a plasma concentration of the opioid receptor ligand from about 200 pg/ml to about 4,500 pg/ml.

19. The method of claim 15, wherein the dissolution profile correlates to a plasma concentration of a metabolite of the opioid receptor ligand from about 20 pg/ml to about 500 pg/ml.

20. The method of claim 19, wherein the metabolite is norbuprenorphine.

21. The method of claim 1, wherein the tensile strength of the device ranges from about 10,000 g/cm² to about 110,000 g/cm².

22. The method of claim 21, wherein the tensile strength of the device ranges from about 30,000 g/cm² to about 100,000 g/cm².

23. The method of claim 21, wherein the tensile strength of the device ranges from about 45,000 g/cm² to about 80,000 g/cm².

24. The method of claim 21, wherein the tensile strength of the device ranges from about 75,000 g/cm² to about 110,000 g/cm².

25. The method of claim 1, wherein the polymer matrix is adhesive.

26. The method of claim 1, wherein the polymer matrix is non-erodible.

27. The method of claim 1, wherein the polymer matrix comprises ethylene vinyl acetate.

28. The method of claim 1, wherein the device releases a therapeutically effective level of buprenorphine for at least 24 weeks.

29. The method of claim 1, wherein the device provides a plasma concentration of a metabolite of buprenorphine of about 50 pg/ml to about 3,000 pg/ml over a period of at least about 24 weeks.

30. The method of claim 29, wherein the metabolite is norbuprenorphine.
